(19) United States
(12) Patent Application Publication
(10) Pub. No.: US 2022/0401663 A1
(43) Pub. Date: Dec. 22, 2022

(54) COMPACT ATOMIZER

(71) Applicant: Loop Laboratories, LLC, Chicago, IL (US)

(72) Inventor: Scott H. Wilson, Chicago, IL (US)

(21) Appl. No.: 17/352,086

(22) Filed: Jun. 18, 2021

(51) Int. Cl. A61M 11/00; B65D 83/28; B65D 83/20

(52) U.S. Cl. CPC A61M 11/007; B65D 83/28; B65D 83/205

(57) ABSTRACT

The present invention comprises a compact atomizer that substantially encapsulates a standard inhalant canister and allows for in-line dispensing through a mouthpiece formed in an actuating cover. The compact atomizer comprises an actuating cover and housing that cooperate to secure the canister. By compressing the actuating cover and housing together, a nozzle of the canister is depressed to release a portion of substance within the canister. Compression is enabled by an actuation surface formed in the actuating cover. Substance is released in-line with the canister through a mouthpiece at the end of the actuating cover. The result is a small, compact, yet attractive atomizer that can be carried in a pocket or the like while keeping the canister free of debris.

### TECHNICAL FIELD

This invention relates generally to inhalant devices, and more specifically, for devices used inhale small, quick doses of atomized liquid, whether for medicinal or recreational purposes.

### BACKGROUND

Inhaling medicine has long been one of the least invasive forms of introducing a substance into the human body. Inhaling immediately introduces the substance to the respiratory system, which quickly transfers it to all parts of the body without a needle, as required to introduce directly to the circulatory system, and without the wait time involved with introducing through the digestive system. This form of intake is also particularly helpful for maladies affecting the lungs, such as asthma.

Inhalers are well known devices that have been used for years, but that have seen little improvement or adjustment. U.S. App. No. 2003/0098024 (“Hodson”) shows a typical inhaler/atomizer. This traditional design involves placing a canister of pressurized liquid (element 107 in Hodson) upside-down into an open cavity. Inside the cavity, a nozzle (111, as shown in FIG. 9 of Hodson) seats into a chamber that holds the nozzle in place when a user presses down on the top of the cannister. This causes a short, controlled release of the pressurized liquid, which quickly expands into a mist in an expansion chamber within the device. The mist is directed typically at a 75-90 degree angle into a mouthpiece that a user has placed in their mouth to receive and breath in the mist.

While inhalers vary in some respects, the vast majority of them take on this traditional design, such as shown in FIG. 9 of Hodson. The design, though effective, is not ideal. First, the open nature of the top end, which is required for the user to press directly on the canister, allows for dust, dirt or other material to enter the cavity. Inhalers are typically carried on the person, such as in a pocket, for easy access. Pockets are hardly clean environments, and simple movements can lead to accidental discharge when the canister is pressed. The typical solution to this issue is to keep the inhaler in a case, but that makes it harder to fit into a pocket and increases the time it takes to access the device. Even without a case, the device is bulky and uncomfortable, with its near right-angle turn and various edges.

### SUMMARY OF THE INVENTION

The present invention addresses these issues by providing a relatively simple, yet unique atomizer design that has evaded the art for decades. The inventors have developed a compact atomizer that avoids the hard right-turn customary with inhalers, thereby reducing the overall size. Though the atomizer distributes the canister contents in-line with the canister, it still provides for a mechanism to compress the canister for actuation without leaving the canister open to the environment. A simple outer cap over the top leaves the device looking indistinct, similar to a tube of lipstick, and with no hard edges. Accordingly, the atomizer may easily be stored in a pocket without a case, and with very reduced risk of inadvertent discharge.

In some instances, the invention comprises a compact atomizer having a canister containing a substance to be inhaled, where the canister has an elongated body with a central axis, a top end, a bottom end, and a nozzle along the central axis through which the substance may be released at the top end. The compact atomizer also has a base platform configured to receive and surround the bottom end of the canister, a sleeve configured to fit around a circumference of the base plate and extend at least a portion of the way along the elongated body of the canister, and an actuating cover configured to fit over the top end of the canister and extend down around at least a portion of the elongated body. The actuating cover has an expansion chamber to receive the substance released through the nozzle of the canister. The base platform, sleeve and actuating cover cooperate to fully encapsulate the canister other than through the expansion chamber.

In other instances, the invention comprises a system for controlling the release of a substance to be inhaled from within a pressurized canister containing the substance, where the system has a base platform configured to receive and surround a bottom end of the canister, a sleeve configured to fit around a circumference of the base plate and extend at least a portion of the way along a central axis of the canister, and an actuating cover configured to fit over a top end of the canister and extend down into the sleeve. The actuating cover has an expansion chamber to receive the substance released from a nozzle at the top end of the canister. The base platform, sleeve and actuating cover cooperate to fully encapsulate the canister other than through the expansion chamber.

### BRIEF DESCRIPTION OF THE DRAWINGS

The present invention can be better understood with reference to the following drawings. The components in the drawings are not necessarily to scale, emphasis instead being placed upon clearly illustrating the principles of the present invention. Where dimensions are provided, they are used for reference and understanding, and are not limiting unless the feature in question expressly claimed to be of a particular dimension. In the drawings, like reference numerals designate corresponding parts throughout the several views.

FIG. 1 is a perspective view of a compact atomizer, showing portions of the front, right side and top thereof, in accordance with a particular embodiment of the present invention.

FIG. 2 is an exploded view of the primary independent and separable components of the compact atomizer illustrated in FIG. 1.

FIG. 3 shows a typical inhalant canister that houses the pressurized liquid to be inhaled.

FIG. 4 is a perspective view of a base component of the compact atomizer of FIG. 1, showing portions of the front, right side and top thereof.

FIG. 5 is a perspective view of an actuating cover component of the compact atomizer of FIG. 1, showing portions of the front, right side and top thereof.

FIG. 6 is a section view of the assembled compact atomizer of FIG. 1, taken through section A as shown in FIG. 1, and with the actuating cover in the unactuated position.

FIG. 7 is a section view of the assembled compact atomizer of FIG. 1, taken through section A as shown in FIG. 1, and with the actuating cover in the actuated position.

FIG. 8 is a perspective view of the compact atomizer of FIG. 1 with an outer cap installed.

FIG. 9 is an exploded view of the compact atomizer of FIG. 1 and the outer cap of FIG. 8 from a perspective view showing the bottoms thereof.

FIG. 10 is a closeup of the section view of FIG. 6, with vent passageways added to introduce ambient air.

FIG. 11 is a perspective view of the upper portion of a compact atomizer with a vented mouthpiece.

### DETAILED DESCRIPTION

The description that follows describes, illustrates and exemplifies one or more particular embodiments of the present invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the present invention is intended to cover all such embodiments that may fall within the scope of the appended claims, either literally or under the doctrine of equivalents.

In a particular embodiment, the compact atomizer invention is comprised of three primary components: a base, a sleeve, and an actuating cover that provides a mouthpiece. The base and sleeve may be referred to collectively as a housing, and may be of unitary construction. These components cooperate to substantially envelop and house a standard inhalant canister. An outer cap may be added to cover over the actuating cover.

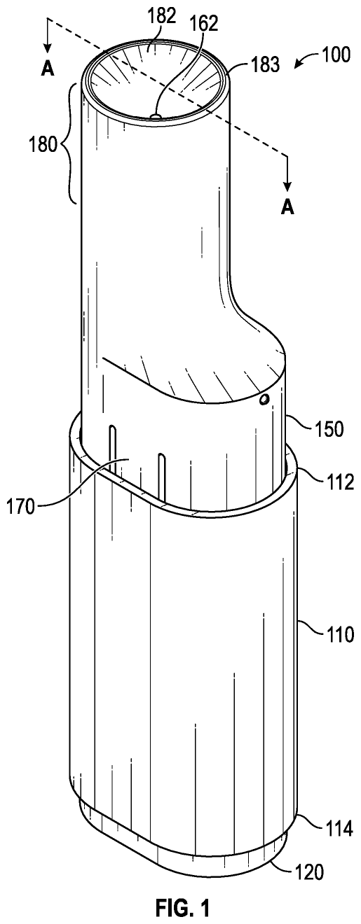

FIG. 1 shows an assembled compact atomizer 100 from a perspective view in accordance with particular embodiments of the present invention. As shown, the atomizer 100 extends from a base platform 120, along a sleeve 110 and up an actuating cover 150 to a mouthpiece 180 at the top of the actuating cover. Housed inside, and not shown in FIG. 1, is a standard aerosol canister 130 containing a liquid inhalant under pressure that expands into a mist upon release. The invention is described herein directionally from the frame of reference of a canister when properly installed within the atomizer. Further, components are described individually and in relation to one another from the frame of reference where the atomizer is sitting on the base platform 120. Thus the “bottom” of components are those portions closer to the surface on which the base platform rests and the “top” of components are those portions further away from said surface, and closer to the mouthpiece upper rim 183, which forms the “top” of the overall device as shown.

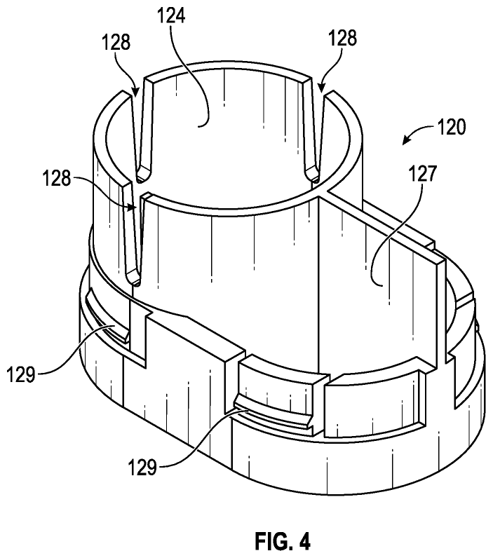

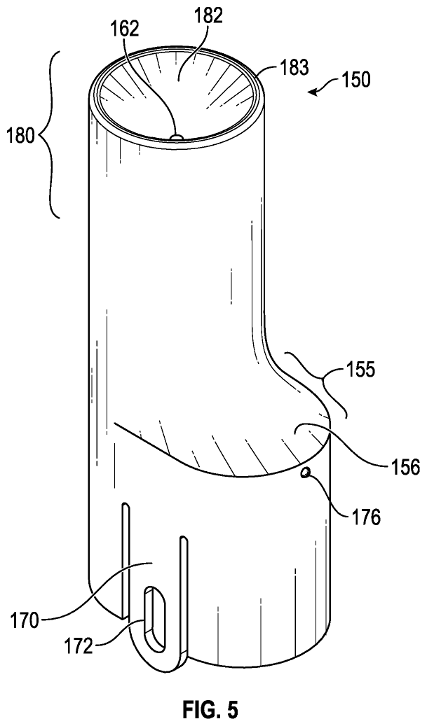

The sleeve 110 is generally hollow, and configured to receive the base platform 120 through an open bottom end 114 and the actuating cover 150 through an open top end 112. The sleeve is generally oval in shape, to accommodate both the cylindrical canister and a lateral extension 155 of the actuating cover 150. As will be further discussed, this lateral extension provides an actuating surface used as a trigger to release inhalant substance from the canister. The actuating cover is further discussed in association with FIG. 5 below, and the base platform is further discussed in association with FIG. 4 below.

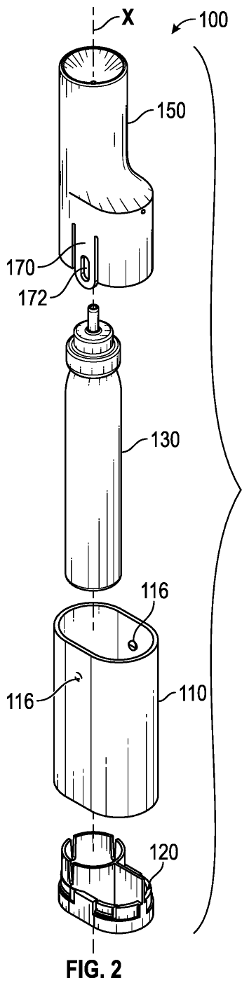

FIG. 2 shows these primary components in an exploded view, along with a standard aerosol canister 130. While the components can be sized to accommodate various sizes of aerosol canister, a common size is no more than a couple of inches in length and a half inch or less in diameter. The components are exploded along a central axis of the canister, which is shown and identified with an “X”. When the term “central axis” or “canister axis” is used within this description, this is the axis being referred to. One of the key characteristics of the illustrated embodiment is that the mouthpiece is aligned with this central axis, and release of the substance within the canister is also along this axis. As discussed above, this allows for a more compact and efficient design than traditional dispensers that involve dispensing at an angle from this axis.

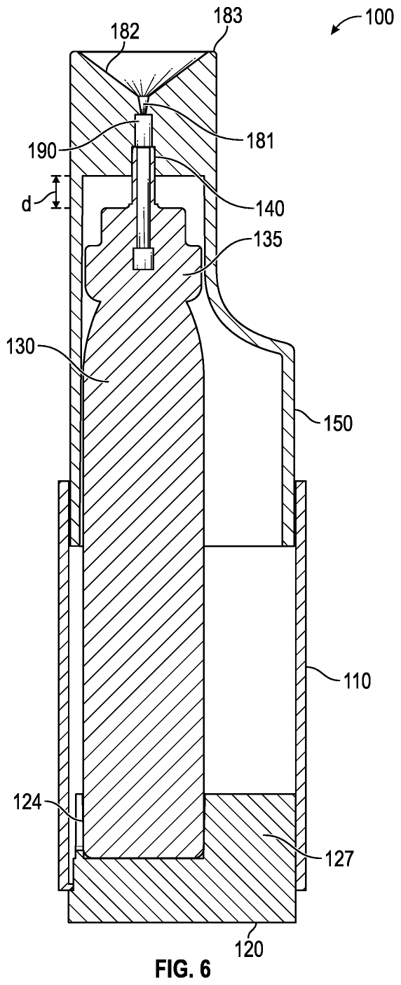

As indicated in FIG. 2, sleeve 110 fits down over base platform 120, and canister 130 is dropped in through sleeve to align with and seat into the base platform. As is clear from the section view in FIG. 6, the canister 130 extends above the sleeve 110 when fully assembled, so it is easy to pull out a spent canister and replace it with a new one. The sleeve provides two detents 116 on its inner surface that protrude inward and align with the slots 172 formed in depressible tabs 170 on the actuating cover 150. Once the actuating cover 150 is placed over the canister and slid down into the sleeve 110, the depressible tabs 170 slide past the detents until the detents 116 pop out in the slots 172. This arrangement serves to keep the actuating cover 150 in place until a user manually squeezes the depressible tabs 170 together, thereby dislodging the detents 116 from the slots 172 and allowing the actuating cover 150 to be removed for canister servicing. The slots 172 also allow for the actuating cover 150 to move relative to the sleeve and base (collectively, the housing), which is necessary to release substance from the canister, as further described below.

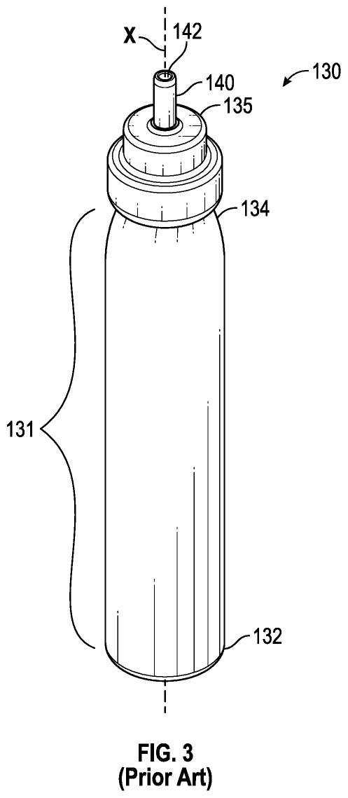

The canister 130 is shown in more detail in FIG. 3. It is designated as “prior art,” because the general construction of the canister is well known in the art. The canister 130 comprises a storage cylinder 131 that extends from a bottom end 132 to a top end 134. Once again, the canister is shown along its central axis “X”. The canister is typically aluminum or rigid plastic that can contain pressurized contents without leaking. At the top end 134 is a cap 135 secured during a filling assembly and not intended for removal by a user. Typically the canisters are used until expended and then discarded in favor a new canister.

Extending from the center of the cap along the central axis X of the canister is a nozzle 140. In some traditional designs, the nozzle may extend laterally out of the canister, or may have a lateral port that releases the contained substance in a direction lateral to the central axis of the canister. But the present design is intended for use with canister 130, which has a nozzle 140 in line with the canister's storage cylinder 131, and a port 142 at the end of the nozzle also in line with the storage cylinder along center axis X. When the nozzle 140 is compressed into the canister 130, a valve is briefly opened within the canister allowing a short burst of substance to enter the nozzle and release out the port 142, pushed forth by the pressure within the canister 130. This burst comprises a metered dose of substance, such that an approximately equivalent amount will be dispensed each time the nozzle is depressed, regardless of how long the nozzle is held in the depressed position.

FIG. 4 shows a perspective view of the base platform assembly 120, which forms the bottom of the atomizer 100. The base platform assembly may be of unitary construction, and made of plastic, rubber, or other moldable material commonly known in the art. The base 120 features a cylindrical female receptacle 124 configured to receive the bottom end 132 of the canister's storage cylinder 131. The receptacle 124 has side walls that extend upward around the canister preferably at least 10% of the length of the storage cylinder 131 when the canister is positioned in the receptacle. It is intended that the canister 130 be easily removable from the receptacle 124 so that the canister can be replaced when empty. Accordingly, the side walls feature slots 128 to better allow the side walls to flex so as to receive and release the canister cylinder 131. A spline 127 extends away from the receptacle 124 in one direction to provide additional stability and structure to the overall atomizer 100.

During assembly, the bottom end 114 of sleeve 110 fits around the outside of the receptacle 124 and the spline 127, and seats around an exterior surface of the base platform 120. Clips 129 positioned around the exterior surface of the base platform 120 fit into grooves formed on the inside of the sleeve 110 (not shown) to hold the sleeve 110 in place. In some embodiments, the sleeve 110 and base platform 120 may be formed of a unitary construction (i.e., a single piece). Whether unitary or formed separately, they form a housing for the canister when assembled as shown and discussed above.

FIG. 5 shows a closer view of the actuating cover 150. Once again, this may be molded or otherwise formed as a unitary construction or assembled from smaller components. The actuating cover features a mouthpiece 180 at its top end. The mouthpiece is simply the portion of the atomizer 100 intended to be placed in a user's mouth to receive a dosage. The mouthpiece 180 features an upper rim 183 that forms the top most portion of the actuating cover. The upper rim 183 is cylindrical in shape. Inside the cylinder, the surface of the actuating cover 150 forms the outlet for substance dispensed from the nozzle 140 of the canister 130 stored within. The outlet is broken into an upper conical outlet 182 and a lower conical outlet 181, which cooperate to gradually allow the substance to expand further from liquid to gas or mist as it exits the pressurized canister 130 through the central hole 162.

Moving along the actuating cover 150 away from the mouthpiece, 180, the exterior wall of the cover 150 expands on one side as a lateral extension 155 that provides an actuating shelf 156. This relatively flat surface can be used, such as with a finger or thumb, to compress the actuating cover toward the base platform 120, as described below. However, ideally, the edges are curved so as to avoid sharp corners on the device. Just below the actuating shelf 156 is another detent 176, this one protruding from the sidewall of the actuating cover 150. In cooperation with another detent on the opposite side, these cooperate to secure an outer cap 200 that may be placed over the actuating cover and butted up against the shell 110.

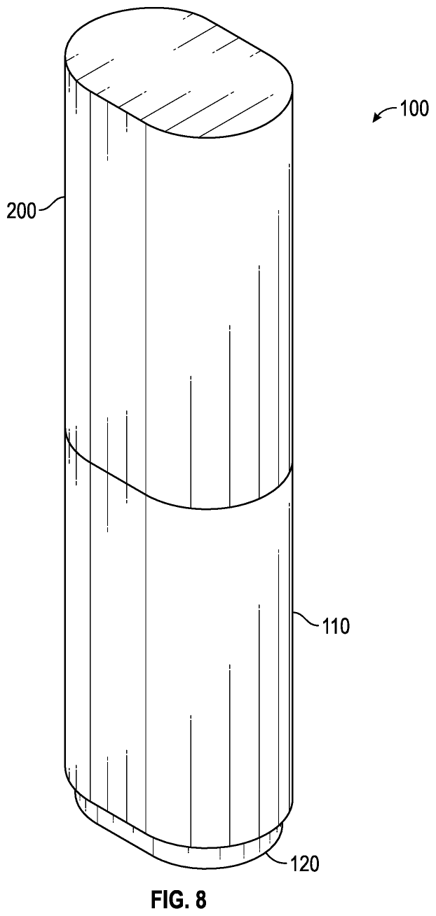

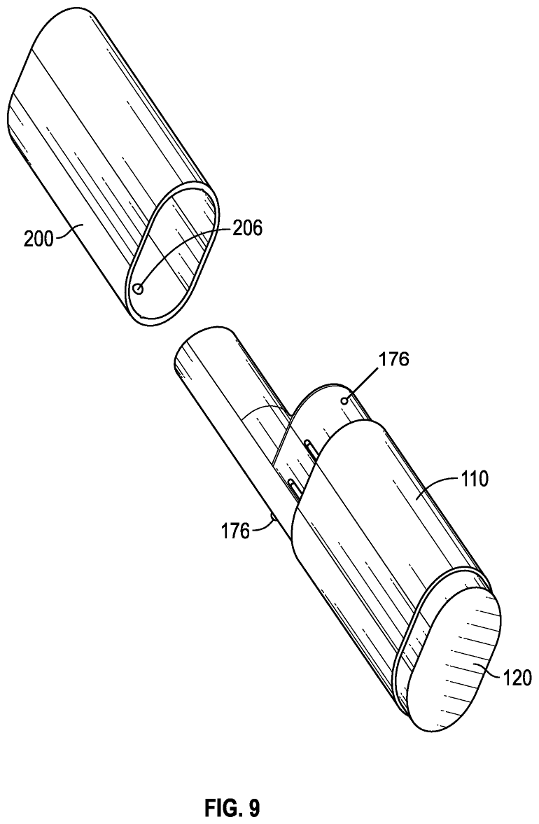

FIG. 8 illustrates the compact atomizer 100 with an outer cap 200 installed, and FIG. 9 shows an exploded view of the outer cap 200 extended away and above the compact atomizer 100. Small pockets 205 bored into the inner surface of the outer cap receive the detents 176 when the cap 200 is pressed over the top of the atomizer 100 to hold the cap in place. When the outer cover 200 is in place, the device looks inconspicuous and does not reveal its function as an atomizer. Further, it can be placed in a pocket or container without risk or concern relating to inadvertent release from the canister or infiltration of dirt or dust.

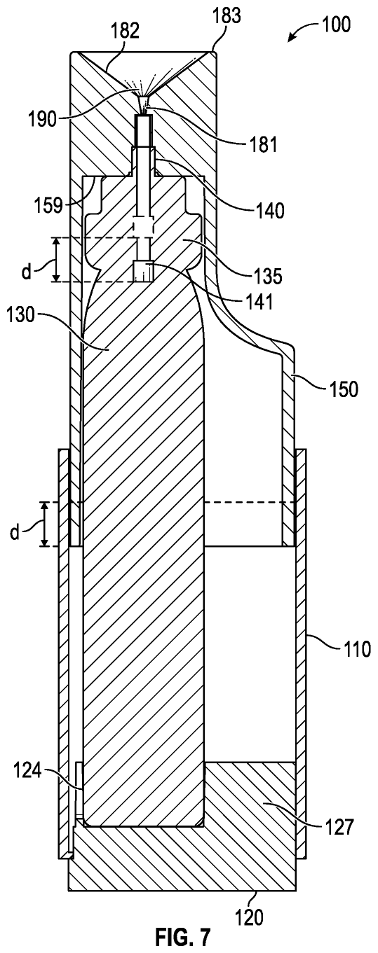

As shown in FIG. 2 and other figures, the actuating cover serves to cover over the top of the canister 130 and its nozzle 140. The cover 150 “actuates” between two positions, which are demonstrated in the section views of FIGS. 6 and 7. In FIG. 6, the actuating cover 150 is in its unactuated, or free, position. In this position, the nozzle of the canister is closed and no substance is being dispensed. By compressing the actuating cover 150 toward the base assembly 120, the actuating cover 150 moves to its actuated position (see FIG. 7). Here, the nozzle is depressed down into the canister to trigger release of a metered dose of substance up through the nozzle and into an expansion chamber 190 situated inside the actuating cover 150 just below the mouthpiece 180. In the expansion chamber, the compressed liquid from the canister 130 is allowed to initially and rapidly expand, causing it to turn to an aerosol mix. This process is then repeated as the contents quickly move toward lower pressure through the lower and upper conical outlets 181, 182 of the mouthpiece.

Typically, the movement of the actuating cover 150 from unactuated to actuated is about 4 millimeters, however it can be more or less than this. As shown, the dimension of allowable travel is designated as dimension “d,” and is limited by the space between the top of the canister cap 135 and a bottom internal surface 159 formed within the actuating cover 150 when the actuating cover is in its unactuated position. This position is, in turn, controlled at least in part by the upper limit of slots 172. In the absence of such a surface, the downward travel (dimension “d”) could be set by the length of slots 172 as detents 116 travel down along them.

As shown, by applying pressure to push the actuating cover 150 down toward the base platform 120 (i.e., into the canister housing), the dimension “d” disappears from its position shown in FIG. 6 as surface 159 sinks down and contacts the top of the cap 135. Meanwhile, the actuating cover 150 sinks “d” further down into the housing/shell 110, and the piston 141 at the lower end of the nozzle 190 is pressed “d” down further into the canister 130, which triggers the release of a metered dose of substance from the canister cylinder 131.

Notably, throughout actuation of the actuation cover 150, the nozzle port 142 never dislodges from the bottom end of the expansion chamber 190. In fact, it is the sidewalls of the expansion chamber 190 that transfer the downward force applied by a user from the actuation surface 156 to the nozzle 140. In this manner, while the actuating cover 150 moves down relative to the canister, the nozzle 140 moves with the actuating cover 150.

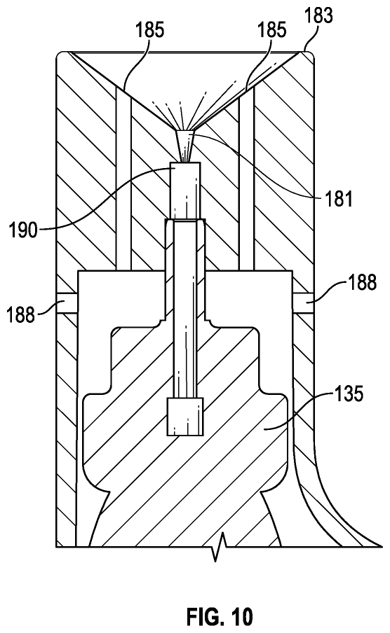

FIG. 10 is a close up of the section view of FIG. 6 that shows the addition of vents to allow entry of ambient air along with the release of substance from the canister 130. Depending on the intended use of the atomizer 100, a mix of ambient air may be needed to help a user inhale the released substance deep into the lungs. As shown, the venting system comprises side vents 188 that allow air to be drawn in around the top of the canister cap 135, as well as mouthpiece vents 185 that allow the air to be drawn up through the mouthpiece 180 and into a user's mouth along with the substance exiting the expansion chamber 190. Notably, these vents provide for an air pathway that does not come into contact with the expansion chamber 190

These vents may be sized and shaped so as to increase or decrease ambient airflow based on the desired function of the device. In some embodiments, the side vents 188 may have user-controllable doors that can be manipulated so as to increase or decrease the ambient airflow, such as by sliding a lever along the outside of the actuating cover. Though side vents 188 are shown around the circumference of the actuating cover 150 near the mouthpiece 180, more vents could be added at other locations to allow the intake of ambient air, such as through the base platform 120 or along the outwardly extended side of the shell 120. Additionally, the vents could be replaced (or supplemented) by a separate air tube running along the side of the actuating cover 150. This tube could be molded together with the actuating cover 150, or floating separately and attached after the actuating cover 150 is formed. So long as the tube has a first end near the mouthpiece 180 such that it is placed in a user's mouth with the mouthpiece, and a second end extending outside of the actuating cover 150 so as to draw in ambient air, the tube will work to bring in supplemental airflow and obtain the deeper lung penetration that may be desired.

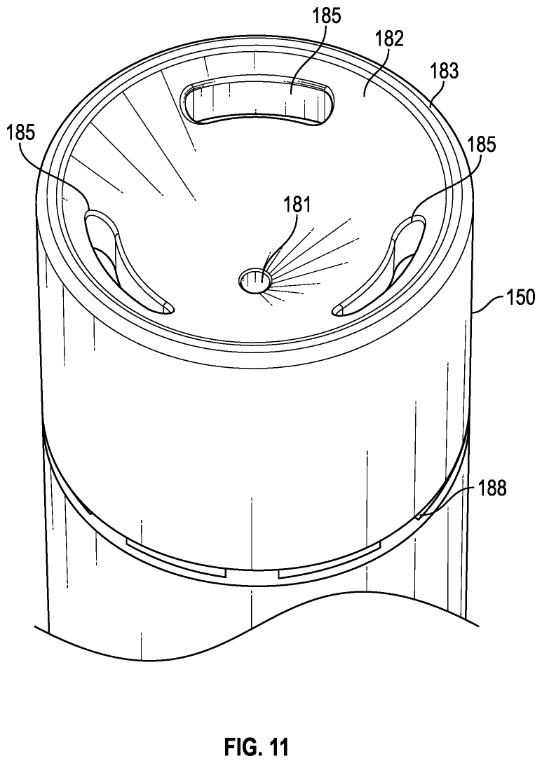

As shown, the vents 188 are vertically positioned so that they remain in the recessed area of the cap 135 when the actuating cover 150 is in its actuated position. If located lower, airflow could be constrained depending on the difference between the maximum cap width and the inner walls of the actuating cover 150. At the same time, it is preferable not to have the vents 188 to close to the mouthpiece 180 because they will not function properly if positioned inside the user's mouth. As shown in FIG. 10, the vents 188 will be below the nozzle 140 extension from the canister 130 when the actuating cover 150 is actuated—which is also when a user would be inhaling to draw air through the vents 188. FIG. 11 is a perspective view of the upper portion of an actuating cover 150, showing how the side vents 188 and mouthpiece vents 185 of section view in FIG. 10 might appear from the outside.

Thus, in accordance with a particular embodiment of the present invention, a user may quickly disassemble and fill the compact atomizer 100 with a new cartridge 130 by simply grasping the device and squeezing the sides of the actuating cover 150 with one hand while pulling down on the housing with the other so as to release the detents 116 from the slots 172. A fresh canister 130 is placed in the receptacle 124, and the actuating cover 150 is installed over the top of the canister 130 and clipped into the housing. To use the atomizer, a user places the mouthpiece 180 into her mouth, places her thumb on the actuation surface 156, and one or more fingers underneath the base platform 120, and then squeezes thumb and fingers together. This action presses the nozzle 140 into the canister 130, ejecting a metered dosage of substance into the expansion chamber 190 of the actuating cover 150 and out through the conical outlets 181, 182 into the user's mouth as an aerosolized spray. For deeper inhalation, the user may simultaneously inhale, thereby pulling ambient air into the mouthpiece through vents 188 in the atomizer connected to mouthpiece vents 185. Releasing the actuation surface will cause the actuating cover 150 to return to its normal position, reloading the atomizer 100 for its next dose. When done, the user may take the outer cap 200, place it over the actuating cover 150, and return the compact atomizer 100 to her pocket or other convenient and discrete carrying location.

Those of skill in the art will appreciate that the features of this invention include, but are not limited to: in-line actuation (i.e., the canister nozzle distributes a dose directly in line with the canister allowing for a more compact construction with fewer hard edges); a substantially encapsulated canister that is protected from the environment; an actuating top cover with a built in mouthpiece; a simple, one-handed dispensing capability; an inconspicuous design; and venting to allow for deep lung penetration.

It will be understood by those skilled in the art that various changes may be made and equivalents may be substituted without departing from the scope of systems and methods disclosed in this application. For example, various methods of formation could be used to create the individual components, which could be formed of plastic, metal, ceramic or other substances suitable for oral usage. Vents could be in different locations and of different quantities. The size of the overall device could be modified to accommodate different canister geometries and sizes. Different mechanisms other than detents could be used to secure the parts together, such as, for example, screw threads, hooks, clips, or a press fit/interference seal. One of skill in the art will understand that these minor design variations could be accommodated and modified from that which is shown in the illustrated embodiments without departing from the concepts claimed below and enabled herein. The selection of material will typically be driven by cost, which may fluctuate making one material more preferable than another from time to time. In addition, many modifications may be made to adapt a particular situation or material to the teachings of the novel techniques without departing from its scope. Therefore, it is intended that the novel techniques not be limited to the particular techniques disclosed, but that they include all techniques falling within the scope of the appended claims.

What is claimed is:

1. A compact atomizer comprising:
a canister containing a substance to be inhaled, the canister comprising a top end, a bottom end, an elongated body extending between the top end and the bottom end along a central axis, and a nozzle at the top end through which the substance may be released in line with the central axis;
a housing configured to receive and surround at least the bottom end of the canister; and
an actuating cover configured to fit over the top end of the canister and nozzle and extend down around at least a portion of the elongated body, the actuating cover comprising a mouthpiece and an expansion chamber positioned along the central axis of the canister and configured to receive the substance when released through the nozzle.

2. The compact atomizer of claim 1, wherein pressing the actuating cover toward the housing causes a controlled release of the substance through the nozzle in a direction along the central axis.

3. The compact atomizer of claim 1, wherein the actuating cover is configured to travel between an unactuated position where it is more distal from the bottom end of the canister, to an actuated position where it is more proximate to the bottom end of the canister.

4. The compact atomizer of claim 3, wherein the mouthpiece comprises an outlet along the central axis through which substance is released in a direction opposite travel of the actuating cover as the actuating cover is moved to the actuated position.

5. The compact atomizer of claim 4, wherein the actuating cover further comprises a lateral extension along at least one side that extends laterally away from the mouthpiece to provide an actuation surface for use in moving the actuating cover into the actuated position.

6. The compact atomizer of claim 1, wherein the housing and the actuating cover cooperate to substantially encapsulate the canister.

7. The compact atomizer of claim 6, wherein the actuating cover is configured to be partially inserted into a top end of the housing and has at least one depressible tab, the tab having a slot that receives a detent formed on an inner surface of the housing.

8. The compact atomizer of claim 7, wherein the actuating cover may be removed from the housing by depressing the depressible tab so as to disengage it from the detent, thereby allowing the canister to be replaced.

9. The compact atomizer of claim 2, wherein the actuating cover further comprises a receptacle for a distal end of the nozzle, the housing having an upper shelf that seats against a top orifice of the nozzle such that, when the actuating cover is moved to the actuated position, the nozzle is pressed into the canister, thereby releasing substance through the nozzle into the expansion chamber in the same direction as the central axis of the canister.

10. The compact atomizer of claim 1, wherein the actuating cover further comprises at least one vent that provides a channel for the passage of outside air through the actuating cover, and wherein said channel does not pass through the expansion chamber.

11. The compact atomizer of claim 10, wherein the channel enters the actuating cover below the canister nozzle and exits through the mouthpiece.

12. A system for controlling the release of a substance to be inhaled from within a pressurized canister that contains the substance, the system comprising:
a housing configured to receive and surround at least a bottom end of the canister; and
an actuating cover configured to fit over a top end of the canister; the actuating cover comprising:
a mouthpiece; and
an expansion chamber positioned between the mouthpiece and the top end of the canister along the central axis of the canister, the expansion chamber configured to receive the substance when released from a nozzle of the canister.

13. The system of claim 12, wherein the actuating cover extends down into the housing and cooperates with the housing to substantially encapsulate the canister.

14. The system of claim 13, wherein the actuating cover further comprises at least one depressible tab, the tab having a slot that receives a detent formed on the inner surface of the housing.

15. The system of claim 14, wherein the detent slides between a top end of the slot when the actuating cover is in an unactuated position and a bottom end of the slot when the actuating cover is in an actuated position.

16. The system of claim 12, wherein the actuating cover is configured to travel between an unactuated position where it is more distal from a bottom end of the housing, to an actuated position where it is more proximate to a bottom end of the housing.

17. The system of claim 16, wherein placing the actuating cover in the actuated position causes substance to release into the expansion chamber along the central axis of the canister.

18. The system of claim 17, wherein the actuating cover further comprises a lateral extension along at least one side that extends laterally away from the central axis of the canister, thereby providing an actuation surface for use in moving the actuating cover into the actuated position.

19. The system of claim 12, wherein the actuating cover further comprises a mouthpiece, the mouthpiece comprising a conical outlet that reduces in diameter along the central axis of the canister from an upper rim of the actuating cover to an outlet of the expansion chamber.

20. The system of claim 19, wherein the mouthpiece further comprises at least one vent that allows for the intake of ambient air that does not pass through the expansion chamber.

21. The system of claim 12, wherein the actuating cover further comprises a housing for a distal end of the nozzle, the housing having an upper shelf that seats against a top orifice of the nozzle such that, when the actuating cover is moved to the actuated position, the nozzle is compressed into the canister, thereby releasing substance through the nozzle into the expansion chamber.
